(19) United States
(12) Patent Application Publication
(10) Pub. No.: US 2023/0402168 A1
(43) Pub. Date: Dec. 14, 2023

(54) MEDICAL SYSTEMS AND METHODS

(71) Applicant: Binx Health Limited, Trowbridge (GB)

(72) Inventor: Jeffrey Luber, Cambridge, MA (US)

(21) Appl. No.: 18/334,463

(22) Filed: Jun. 14, 2023

Related U.S. Application Data
(60) Provisional application No. 63/352,047, filed on Jun. 14, 2022.

(51) Int. Cl. G16H 40/20; G16H 80/00; G16H 10/60; G16H 10/40; G06Q 40/08

(52) U.S. Cl. CPC G16H 40/20; G16H 80/00; G16H 10/60; G16H 10/40; G06Q 40/08

(57) ABSTRACT

The invention provides computer-based systems by which a physician may consider information or a request from a patient and order a test or procedure. A patient may make a request for medical services and the system allows a physician to consider the request and even order at least a portion of the medical services within the system. The system records the initial request and order as a visit in a form and manner consistent with requirement of a healthcare organization.

### TECHNICAL FIELD

The invention relates to computer-based systems with a smartphone app or similar interface by which a patient may order a medical test or service.

### BACKGROUND

Delivery of healthcare is impeded by hard-to-reach, resistant, and non-compliant segments of the populace. Some people live in remote or rural locations and find it difficult to visit a doctor's office. Some people for personal reasons are simply reluctant or unwilling to visit a clinic. Nevertheless, people may still get sick or injured, and it is well understood that prompt testing and response is essential to successful treatment and recovery.

Unfortunately, the delivery of medical services is commonly limited by the paperwork and formalities required by hospital systems and insurance companies. Many hospital systems will not allow an ailing person to get past a “phone tree” and talk to a human medical provider without entering a valid insurer ID and group number. Thus, despite the potential availability of a live human doctor being ready to help people suffering from injury or sickness, the bureaucracy of the healthcare industry largely excludes large segments of the population, such as remote, low-income, underemployed, and underinsured segments of the population from even having an initial visit with a doctor.

### SUMMARY

The invention provides computer-based systems by which a person may use a smartphone app or similar to request a medical service from his or her selected provider or care team, and have the provider review the request and order at least part of the medical service, such as a test. The patient need not travel in-person to see the provider, at least for the initial test or service. Instead, the provider can review any information provided by the patient and order a test or procedure. The request is handled, and the test is ordered, by the person's existing or chosen provider or care team. Thus, when a person or family has been, for example, seeing a family doctor for some time, and that provider has relevant knowledge and records of the medical background, by using the smartphone app, the person can send the request to that chosen provider or care team. The smartphone app sends the request to a back-end computer system for review by the person's chosen provider. That provider orders the test and a fulfillment lab or similar contracted test provider promptly sends the test and/or relevant instructions to the person.

For a test that uses a sample that the patient can collect at home, such as a cheek or nasal swab, or a urine sample, for example, the contracted test provider may immediately ship the sample collection kit to the patient. The patient makes the initial request via a smartphone app or similar, the chosen provider orders the appropriate test, and the relevant kit or instructions are sent immediately to the patient. A sample collection kit may be shipped to the patient along with a return shipping label for sending the sample to the contracted test provider. Or, for certain test types, the patient may be instructed to visit a local clinical services lab, e.g., for a blood draw, which may then be shipped to the contracted test provider. The contracted test provider performs the assays and analyses to obtain results that are loaded into the back-end computer system for professional review by the chosen provider or care team.

A sick or injured patient need only use a smartphone app or similar to have the appropriate test ordered and then to receive the kit or instructions that will be used to get test results for use by the chosen provider for diagnosis and treatment. From the patient's point of view, seeking treatment is as simple as pushing a button on a smartphone app. Behind the scenes, backend computer systems of the invention coordinate activities of the chosen provider or care team, contracted testing laboratories, and hospital systems to ensure that the right test is ordered promptly, that the appropriate instructions and materials are sent to the patient, and that the decisions, orders, and instructions of the medical professionals are recorded appropriately in electronic health records or billing records.

An important feature of systems and methods of the disclosure is that a patient or user uses a preferred, chosen provider or care team. For example, when a person has an existing, long-standing relationship with a medical provider, the request, made through the smartphone app, is sent to that medical provider. The backend computer system may be provided or maintained by a third-party service or by the contracted test provider. To use the system, clinics, physicians, providers, or care teams register as participants and may then inform their patients of the availability of the smartphone app or similar and of the person's ability to have their chosen provider or care team order a test at the press-of-a-button. This means that the test is ordered, and results will be reviewed by, the person's preferred provider or care team. The system can work within an existing doctor/patient relationship or within any other existing or chosen relationship that a person has with a medical provider.

Systems of the invention electronically manage patient requests and provider orders, allowing medical services to be conducted, or at least initiated, early in a patient's journey through the healthcare system. A person may make a request for medical services and the system allows a chosen provider or care team to consider the request and even order at least a portion of the medical services within the system. For example, when a patient shares a complaint and requests treatment, a physician may determine that a blood test is required and order a blood draw and test. The system directly instructs the patient to visit a test site with a phlebotomist for a blood draw. The system records the initial request and order as a “visit”. In fact, the system records the visits in a manner consistent with healthcare billing practices, allowing a hospital, clinic, or doctor's office to bill for the visits recorded in the system. The system may create a visit record that includes the patient's insurance information or, in other situations, the system may create the visit record but allow insurance information to be populated later. This allows a doctor to have the patient go for a blood draw or other test before the patient has shown up at the hospital to complete intake paperwork.

Advantageously, systems and methods of the invention allow the provider to order that initial test or service (blood draw, flu shot, etc.) very quickly in response to the person's initial request. Systems of the invention facilitate those requests and orders, record those as visits, and can even employ digital communications platforms that satisfy healthcare organization standards defining what qualifies as a visit. For example, wherein a healthcare organization (e.g., Medicaid) only recognizes as visits those interactions that involves synchronous, real-time communication between the physician and patient, the system may employ a digital communications platform (e.g., messaging service or videoconference platform) that satisfies those standards. Regardless of the digital framework provided by computer systems of the invention, from the patient's point of view, the patient uses an app on a smartphone, tablet, or similar person device to report an injury or sickness, and a useful test kit, treatment, or instructions are immediately sent to the patient. Using the app, a patient could report symptoms and be sent a Covid test, or prescription refill, or finger-prick tool, or instructions to go to the closest phlebotomist, etc. When the patient uses the app, behind the scenes, a physician/provider orders the medically-relevant test or service, which is promptly sent to the patient. While the patient using the smartphone typically will not see this, the system may be programmed to use and record diagnostic and/or billing codes for those inquiries and orders. The system benefits patients by providing physicians and clinics with tools by which to quickly respond to patient needs, even remotely, in an environment that may be structured to satisfy what defines a visit according to a governing healthcare organizations or insurance standards.

Systems and methods of the invention provide clinics and hospitals with tools by which a physician may directly and rapidly see a large number and variety of patients. Systems and methods of the invention tools remove various barriers such as geography allowing a doctor to reach and serve under-served populations. The doctor may order tests and treatments very rapidly, allowing patients with a complaint to engage with the healthcare system and be treated very early in the presentation of systems. Due to early engagement, treatment outcomes are improved. Traditionally under-served populations (e.g., rural and remote) are better engaged and addressed, improving healthcare across the fabric of society. Systems and methods of the invention may be designed to record visits according to the requirements of hospital systems, government agencies and/or insurance systems. Recording visits in this manner encourages adoption of the disclosed systems by clinics and hospitals. Where the complexity and arbitrary bureaucracy of government agencies and insurance systems have presented a barrier to physicians engaging with remote, dispersed, unengaged, and otherwise difficult-to-serve populations, systems of the invention overcome those barriers and provide for improved healthcare outcomes across previously-unreachable segments of the population.

In certain aspects, the invention provides a virtual medical service method. The method includes processing, by a server computer system, a request for a medical service (e.g., received from a person's use of a smartphone app or similar), sharing the request with a medical provider, and receiving into the server computer system an order from the medical provider for at least a portion of the medical service. The server computer system informs the patient, via the smartphone app or similar, of the order and records a record of the order therein. The system preferably writes a billing code into the record. The method may include recording a plurality if visit records each day for at least several days and periodically transferring the visit records to a billing system computer. The request may be received when a patient uses a smartphone app that transmits the request to the server computer system.

In some embodiments, the order from the medial provider includes an order for a test and the method includes sending instructions to the patient to go to a test site. The server computer system may send the order to the test site. The method may include facilitating, by the server computer system, the patient and the test site in scheduling the test. For example, the patient may be instructed to go to the test site and give a sample (e.g., of blood, breath, or urine).

The person using the smartphone app may be a patient making a request to his or her physician for a medical test. In but one illustrative example, the person may request a test for a sexually-transmitted infection (STI) by pressing a button on a smartphone app. The person's chosen doctor sees the request and orders the test. A backend computer system that manages the request may then send the order to a contracted testing company, which company then promptly ships, for example, a home test kit to the patient. The home test kit could include, for example a lateral flow assay to be exposed to urine, or a cheek or nasal swab, or other such sample collection device(s), as well as a return label and packaging. The person collects the sample at home and uses the return label and packaging to send the sample to the contracted testing company, who analyzes the sample and posts results to a portal for view by the person's chosen physician. One important benefit of methods and systems of the disclosure is that the person is making the request to a selected or chosen provider or care team. The person can know the identity of the person or team ahead of time. It may be a team of medical providers at the person's regular doctor's office. The person may select a provider based on reputation or due to a referral. The provider or care team has opted into use of the backend computer system and may inform the person of the availability of the smartphone app. Once the person installs the app on his or her phone, ordering the test may be as simple as pressing a button.

Some mentioned examples illustrate medical tests and services such as STI tests, covid tests, prescription refills, and blood tests. Any of a variety of medical and incidental tests and services are within the scope of the disclosure and may make use of the disclosed systems and methods. For example, a person may request dental consultation and services through the platform. A dentist may respond to a request and ship a test kit, a medicine or reagent, or a dental device to the person. Note that the person may be making a request on his or her own behalf, or on behalf of a family member or another person. For example, a person may use a smartphone app to request a medical service on behalf of an elderly or infirm person, and a medical provider may use the backend computer system or order medical transport, or the shipment of a material or device important to the care of the subject. The platform may be used to order, for example, veterinary services. For example, a person could order a test for their dog and have a sample collection kit shipped to the home by use of the smartphone app. Systems and methods of the invention may be particularly beneficial for large animal veterinary services, where it may be difficult to transport, e.g., a horse to the clinic, or where a straightforward prescription refill is requested. In all cases, the user—via the smartphone app (or similar)—may request the medical services from his or her preferred, chosen medical provider or care team, someone with whom they have an existing relationship or are seeking due to reputation or referral.

In certain embodiments, the medical provider and the patient conduct a real-time synchronous discussion by text, voice, or video via a digital communications platform and the computer system receives a confirmation of the synchronous discussion and writes the confirmation of the synchronous discussion into the visit record. The computer system may upload the visit record to an electronic medical record system.

The method may include authorizing, by the server computer system, a laboratory to ship a medical test kit to the person and optionally accepting input of a test result from the laboratory and providing the person or the medical provider with access to the test result. The server computer system may periodically transmit batches of records to an insurance system or to a medical billing specialist within a hospital system or clinic. The computer system may write the visit record as a billable record of a visit to an electronic medical records system. The method may include initiating or ordering, by the computer system, shipment of a medical test kit directly to the patient.

Aspects provide a system that includes at least on processor coupled to a memory subsystem. The memory subsystem includes instructions executable by the processor to cause the system to obtain a digital patient request for medical service, share the request with a medical provider, and receive an order from the medical provider for at least a portion of the medical service. The system informs a patient of the order and writes a record of the order in the computer system. Preferably the system writes a billing code into the record. The system may record a plurality if visit records each day for at least several days and periodically transfer the visit records to a billing system computer. The request may be received when a patient uses a smartphone app that transmits the request to the computer system.

The system may be server- or cloud-based server computer system that provides the patient with access to an app, for download to a smartphone or tablet. The patient can download and install the app and register to begin the process of making the request. From the patient's point of view, once the app is installed and registered, all that is required is using the app to request a medical service or report symptoms. The appropriate test or instructions are promptly sent out to the patient. Behind the scenes, the server system presents the request to a medical provider and allows the medial provider to order a test.

The order from the medial provider may include an order for a test, and the system can send instructions to the patient to go to a test site. The system may send the order to the test site. Optionally, the system instructs the patient to go to the test site and give a sample e.g., of blood, breath, or urine. A digital communications platform may conduct a real-time synchronous discussion between the medical provider and the patient by text, voice, or video and the system may receive a confirmation of the synchronous discussion and write the confirmation of the synchronous discussion into the visit record. The system may upload the visit record to an electronic medical record system. Preferably the system authorizes a laboratory to ship a medical test kit to the person; accepts input of a test result from the laboratory; and provides the person or the medical provider with access to the test result. In some embodiments, the system periodically transmits batches of records to an insurance system. Optionally the system writes the visit record as a billable record of a visit to an electronic medical records system.

### BRIEF DESCRIPTION OF THE DRAWINGS

FIG. 1 shows steps of a virtual medical service method.

FIG. 2 shows a system useful for performing the method.

### DETAILED DESCRIPTION

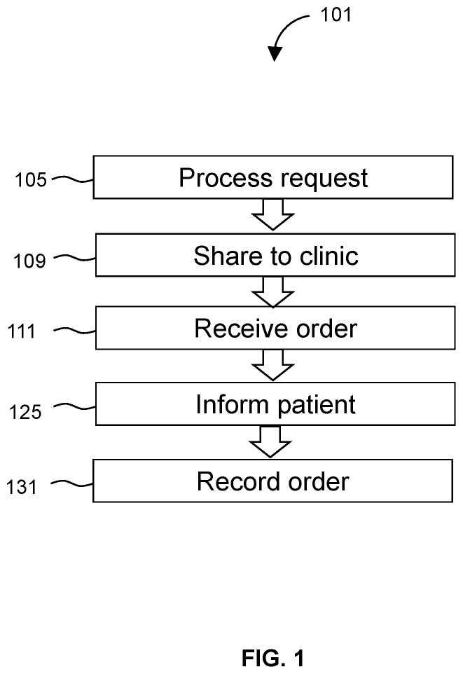

FIG. 1 shows steps of a virtual medical service method 101. The method 101 includes processing 105, by a computer system, a request for a medical service; sharing 109 the request with a medical provider; receiving 111 into the computer system an order from the medical provider for at least a portion of the medical service; informing 125 a patient of the order; and recording 131 a record of the order in the computer system. The method 101 may begin with processing 105 at a computer system a request for a medical service.

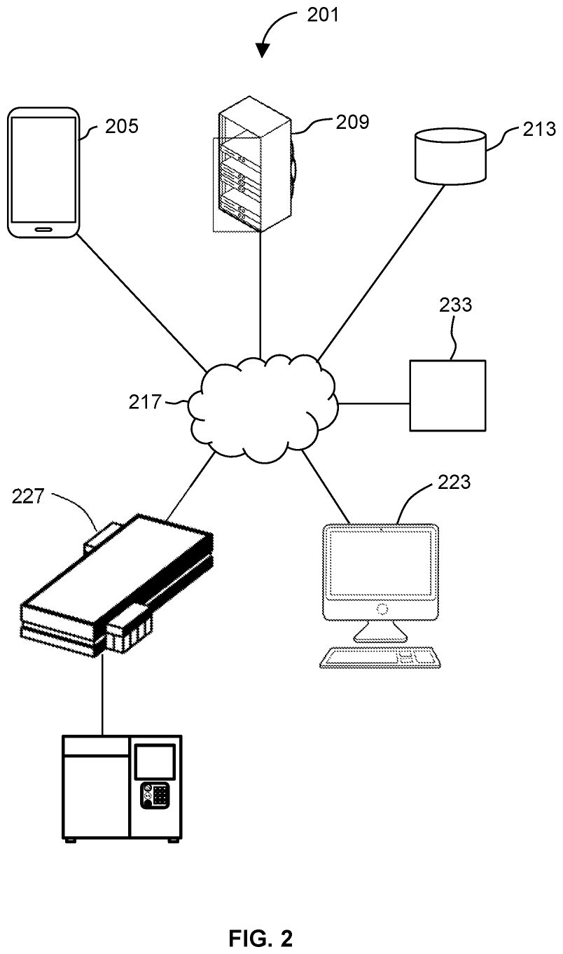

FIG. 2 shows a system 201 useful for performing the method. The system 201 preferably includes a computer system 209 that processes 105 a request received from a patient. The patient may transmit the request using a personal device 205 such as a smartphone. Preferably, the computer system 209 provides the personal device 205 with an ability to download a smartphone app or access a web page and register for use of the system 201. The smartphone app is installed on the personal device 205 and allows the patient to log in securely. The smartphone app or web page allows the patient to send a request for a medical service.

Preferred embodiments may involve activities of a contracted testing company such as a clinical services laboratory 227. For example, the clinical services laboratory may provide the computer system 209 with software and applications to implement the method 101. A medical provider registers to use the computer system 209, e.g., optionally creating an account or registration with the clinical services laboratory 227. The medical provider access the system 201 through the clinic computer 223. Preferably the computer system 209, optionally provided by the clinical services laboratory 227, presents a provider portal via the clinic computer 223. A provider can inform a patient or person of the availability of a smartphone app that operates in the system 201. The person installs the smartphone app on their smartphone 205 or similar personal device. Then the person has a single-button app by which to request a medical test or service from his or her chosen, preferred provider or care team. Information flow may be coordinated by the computer server 209 (which may be any number of servers or cloud resources operating in combination) to receive requests from the smartphone app, receive orders from a medical provider using the clinic computer 223, and cause the clinical services laboratory 227 to ship or send a test kit, device, instructions, or other materials to the person, or to another person or location on whose behalf the requestion person is acting.

The patient may request a medical service via the smartphone app. For example, the patient may input a description of one or more symptoms, and request a visit to have those symptoms considered by the doctor. The patient could request a specific test, such as a test for a virus (e.g., Covid) or a sexually-transmitted infection. The computer system 209 processes the request. The computer system 209 may preferably share 109 the request with a doctor at a clinic or hospital. For example, the doctor may receive the request at a clinic computer 223. Preferably, a telecommunications network 217 passes the patient request from the personal device 205 to the computer system 209. The computer system 209 contains a processor linked to a memory subsystem containing program instructions executable by the processor to process 105 the request and share 109 the request with the clinic via the clinic computer 223.

The computer system 209 preferably includes one or any combination of server computers or cloud computing resources. Server computer generally include rack-mounted computers in any number such as one, a few, or dozens, optionally within a clinic or hospital system and optionally at a fixed location in one or more data centers. Cloud computing resources generally include similar hardware in one or more data centers but generally are not in fixed locations. Cloud computing resources generally include instances of servers or virtual servers that are made available temporarily or on-demand by a third-party service provider operating a very large number of hardware servers. Suitable cloud computing resources include the cloud services offered under the trademark AMAZON WEB SERVICES by Amazon, often referred to as AWS and understood to include on-demand web servers. Steps of the method 101 may be performed by software packages implemented by the servers. Such software may be provided in any suitable code or combination of code including both compiled or non-complied code, developed in any of, e.g., Python, C++, Ruby on Rails, Groovy and Grails, etc.

The clinic computer 223 may be part of a clinic, hospital, or hospital system that also includes or communicates with an electronic medical record system 213 and also optionally a clinical services laboratory 227. For example, the clinical computer 223 and/or the computer system 209 may each communicate directly or indirectly via a communications network 217 with the clinical services laboratory 227, optionally more specifically with a laboratory instrument or a laboratory information management system (LIMS) that directly communicates with the laboratory instrument within the clinical services laboratory 227. Any or all of electronic medical record system 213, clinical services laboratory 227, the hospital system computers, and the laboratory information management system (LIMS) may be provided by any number or combination of server computers and cloud computing resources. The communications network 217 generally includes the hardware by which any of the computer system share data and instructions, which hardware may include any one or combination of the Internet, cell phone networks, switches, firewalls, and other hardware of telecommunications.

The system 201 generally includes or communicates with a number of individual computers including, for example, the personal device 205, the clinic computer 223, any server or cloud resource within any of the computer system 209, the electronic medical record system 213, computer resources of the clinical services laboratory 227, the hospital system computers, and the laboratory information management system (LIMS) computers. Each computer, in general, will include at least one processor coupled to a memory subsystem and one or more input/output devices. A process generally refers to a chip, a computer chip such as the i5 processor from Intel. A memory subsystem generally includes one or a combination of random-access-memory (RAM) and read-only-memory (ROM), which are tangible, non-transitory storage mediums for data, files, and computer program instructions. I/O devices generally include monitors, keyboards, pointing devices (mice and trackpads), Wi-Fi cards, network interface cards (NICs), modems, antennae, and others.

When the request for a medical service is shared 109 with a provider or care team, e.g., at a clinic or hospital, via the clinic computer 223, the provider may review the request and decide to order at a least a portion of the medical service. For example, the provider may order a medical test. The medical test may be something that the patient can perform in private at home, and the provider may order a test kit to be shipped to the patient. The medical test may require that the patient travel to the clinical services laboratory 227. The provider inputs the order into the clinic computer 223 and the computer system 209 receives the order. The computer system will then inform 125 the patient of the order, using the communications network 217 to send information about the ordered service to the patient via the patient's personal device 205. Critically, the computer system 209 records 131 the order in memory, writing a record that the physician has communicated with the patient about the patient's symptoms and/or request and that the physician has, in view of the request and/or reported symptoms, decided to order, and ordered, at least a portion of the medical service that the patient requested.

It may be a common use case that the patient orders a test and one or more visits with the physician and additionally a diagnosis of a condition and treatment for the condition. In such scenarios, the computer system 209 is useful for providing an initial “visit” between the patient and the doctor, allowing the doctor to order an initial test and gather a rich amount of medical information before the patient travels to the doctor's office for an in-person visit with the doctor. In a specific example, the patient may request, via the personal device 205, a Covid test. The computer system 209 processes 105 the request and shares 109 the request with the physician at the clinic. The physician may determine that a PCR test will be the most informative tool for determining what course of treatment is medically-recommended for the patient. The physician may order a PCR test to be performed at a clinical services laboratory 227 and write that order using the clinic computer 223. The computer system 209 receives 111 the order, informs the patient of the order, and records 131 the order in memory.

Thus the computer system 209 includes at least one processor coupled to a memory subsystem containing instructions executable by the processor to cause the system to process 105 a digital patient request for medical service; share 109 the request with a medical provider; receive 111 an order from the medical provider for at least a part of the medical service; inform 125 a patient of the order; and record 131 a record of the order in the computer system. Preferably, the system 209 is programmed to write a billing code into the visit record.

The visit record may be written to an electronic medical record system 213, which may be part of, or connected to, the computer system 209. The computer system 209 is set up to facilitate visits between the physician and patients. For example, each time the physician considers the request from a patient and orders at least a portion of a medical service requested by the patient, the system 209 can record a visit. In fact, the computer system 209 may include instructions that structure how the order is received 111 to satisfy any institutional requirements for what constitutes a visit between a doctor and a patient. For example, some institutions may require that synchronous communication transpire between a doctor and a patient before the communication (e.g., service request and service order) constitute a medical visit. In such situations, the computer system 209 may be programmed to facilitate synchronous communications. For example, the computer system 209 may itself directly provide a communications platform such as for communication via text messages, voice, or video conference. In some embodiments, the computer system 209 is in operable communication with an outside service such as a digital communications platform. For example, the system 209 may provide the patient and the physician with links to a partner digital communications platform such as a voice-over-internet protocol calling service or a videoconferencing platform. In such embodiments, the patient and physician conduct, via the digital communications platform, a real-time synchronous discussion, e.g., by text, voice, or video, and the computer system 209 receives a confirmation of the synchronous discussion and writes the confirmation of the synchronous discussion into the visit record.

It is noted that when the physician orders at least a portion of the service, the computer system 209 may write a billing code to the record. For example, the clinic computer 223 may provide the physician a tool, such as menus or an interactive interface, allowing the physician to order a particular test or service. When a specific test or service is ordered via the clinic computer 223, the computer system 209 may write an appropriate or pre-programmed billing code into the record. The billing code may be pre-programmed within the logic of computer system 209, or the billing code may be selected and/or written in by the physician, or by another medical professional or clinic or hospital staff, such as an intake coordinator, nurse, medical billing specialist, or billing coordinator.

In preferred embodiments, as a result of receiving 111 and recording 131 the order into a visit record, the computer system will have a record of one or any number of visits. The computer system 209 may be programmed so that each visit record includes any one or more of any of the following specific pieces of information: patient identify, chief complaint, description of symptoms, service requested, record of communication between physician and patient, physician ID, date and time and other demographic data, billing codes, and other insurance information (e.g., patient plan and subscriber number).

In certain embodiments, the system 209 uploads the visit record to an electronic medical record system. Preferably, the information included in the visit record is selected to satisfy an institutional definition of a visit that is billable. The institution may be an insurance company, a medical board, a governmental medical services plan (e.g., Medicaid or Medicare), a consortium of insurance companies, or a health system. Such an institution may have a definition of a billable visit that includes certain minimal requirements (such as for live, synchronous, and/or real-time communication between a doctor and patient, specifying whether text, audio, or audio-visual meets the requirement). The computer system 209 can facilitate, or link to a digital communications platform that facilitates, the communication and also write in the visit record that such communication transpired. The computer system 209 may include an interactive form by which the physician signs, e.g., provides an e-signature to, the record, e.g., to validate that the communication transpired. The computer system 209 may further be operable to transfer, e.g., send, the visit record to a billing system computer 233.

The billing system computer 233 may be provided as a component of the computer system 209 or may be a separate, third-party entity. The billing system computer 233 may be, for example, a local network of computers in use within the medical billing department of a hospital system. In some embodiments, the computer system 209 records a plurality if visit records each day for at least several days and periodically transfers the visit records to the billing system computer 233. Periodically may mean hourly, or each evening, or twice daily, or weekly, or monthly, or similar. Using the computer system 209 in this manner allows the physician to visit with a plurality of patient and record and transmit records of visits to the billing system computer 233. Preferably, billing system computer 233 is part of, or communicates with, an insurance system and the computer system 209 periodically transmits batches of records (e.g., visit records) to the insurance system. Preferably, the computer system 209 writes billing codes into the records. The computer system 209 may be operable to write the visit record as a billable record of a visit to an electronic medical records system 213, which may be used to transmit records to the billing system computer 233.

Benefits of the described computer system 209 arise in giving the physician an ability to directly and quickly order at least a portion of a medical service to be provided directly to a patient. For example, when a patient feels concern that he or she may have symptoms of a medical condition, the patient may make a request for a medial test (and, optionally, treatment) via a smartphone app or website on a personal device 205. The patient may place the request without delay, without any requirement of telephoning a hospital to schedule an appointment. The physician, using the clinic computer 223, may review the request as well as multiple others.

Here, the physician has the opportunity to evaluate the priority of each of multiple requests for a medical service. Even for numerous requests for the same medical service (e.g., Covid test), the physician may evaluate the priority of each request against additional information. In fact, the computer system 209 may push out prompts for additional information (e.g., vaccine status, age, cardiovascular history), optionally in real-time under direction of the physician. In some optional embodiments, the physician can issue customized questions or push out a pre-scripted survey during a visit facilitated by the computer system 209. The physician can thus gather additional information and determine, for example, to order the requested tests for each patient, but may flag different requests, and different test orders, with differing priorities. For example, a patient requesting a test for a condition who has one or more co-morbidities for the condition, may be prioritizes and informed 125 that the test has been ordered with priority and instructed to promptly visit a clinical services laboratory 227.

Thus in some embodiments, the order from the medial provider includes an order for a test, and the system 209 sends instructions to the patient to go to a test site such as a clinical service laboratory 227. More specifically, the test site may be a patient-facing location of a larger clinical services laboratory 227. The patient-facing location may be a local site where samples are collected. Sample collection at the local test site may involve any one or more of: a blood draw, a cheek swab, a nasal swab, an Helicobacter pylori or similar breath test, a urine or stool collection, an x-ray or other medical imaging procedures, or any other suitable sample collection or test procedure.

In one example, the patient, via his or her personal device 205, requests treatment with a description of symptoms that could be consistent with kidney disease or diabetes. The physician reviews the request at the clinical computer 223 and determines that a basic metabolic panel will be most informative. The basic metabolic panel (BMP) requires blood collection by phlebotomy followed by laboratory assays for some combination of levels of calcium, glucose, sodium, potassium, bicarbonate, chlorite, blood urea nitrogen, and creatine. The physician orders the BMP test and the computer system 209 informs 125 the patient that the BMP test is ordered. The computer system 209 instructs the patient to go to a test site of a clinical services laboratory 227 where a phlebotomist is available. The computer system 209 also informs the clinical services laboratory 227 that the physician has ordered the BMP test.

In some examples, the computer system 209 works according to visit standards provided by an outside entity such as an insurance consortium or government agency (e.g., Medicaid/Medicare). The visit standards may require, for example, that a billable patient visit requires real-time, synchronous conversation between the patient and the physician. The computer system 209 facilitates the conversation (e.g., the visit) via a linked digital communication platform. Once the visit has occurred, the computer system 209 records the visit and the order for the BMP test, optionally writing one or more diagnostic or billing codes into the visit record.

Other embodiments operate in a similar fashion but, instead of necessarily instructing the patient to visit a clinical services laboratory, may authorize shipment of a medical test kit to the patient. For example, in some examples, the patient may be requesting a Covid test because the patient is required to show a negative Covid test for some real-world condition. For example, the patient may be a college student who must show a negative Covid test to return to the college campus. In such an example, a physician may determine that the patient is relatively low-risk (e.g., already vaccinated and boosted, relatively young, and with no co-morbidities). The physician may determine that the patient's medical needs will be met by a rapid, at-home, antigen test. The patient may place an order to have one or more antigen test kits shipped to the patient. The computer system 209 receives 111 the order, informs 125 the patient, and may also authorize a laboratory or distribution facility to ship a medical test kit to the patient. In this example, the distribution facility ships the ordered number of home antigen tests to the patient. The computer system 209 preferably records that a visit has occurred.

When the test is performed by a clinical services laboratory 227 (e.g., the BMP test example), the computer system 209 may accept input of a test result from the laboratory and provides the patient or the medical provider with access to the test result. In such a manner, the initial request that was received from the patient and processed 105 by the computer system 209 can lead to clinical results quickly that the physician can use to determine next steps of treatment for the patient. Of importance, is that the patient can make the initial request via a personal device 205. The computer system processes 105 the request and shares 109 the request with a physician, e.g., via a clinic computer 223.

This process allows a physician to directly respond to a patient's request and even to directly order at least a portion of a medical service, engaging the patient in healthcare and/or laboratory diagnostic services more rapidly than with physical, in-person visits to a doctor's office. Thus the system 201 provides a platform for visits that can engage a large number of, and wider demographic range of, patients than can be reached by requiring in-person office visits. The system 201 of the disclosure may find particular utility in reaching people in rural locations. Importantly, the computer system 209 is programmed to record 131 patient requests and services ordered as visits, e.g., in visit records. Each visit record may be written to include a diagnostic or billing code.

The computer system 209 may upload the visit record(s) to an electronic medical record system 213. In some embodiments, the computer system periodically transmits batches of records to an insurance system 233. Preferably, the computer system 209 writes the visit record as a billable record of a visit to an electronic medical records system 213, and thus is useful by the physician for practicing medicine within the context of a hospital or health system that has commercial or institutional record-keeping or billing practices and requirements.

Other features that may be provided by the system are push-surveys to personal device 205 to gather information from the patient, shipment orders to a have a test or kit directly shipped to the patient, clinical services authorization (e.g., ordering a test and sending a patient to a laboratory 227 to have a sample collected), tailoring to institutional standards (e.g., writing records that satisfy Medicare or Medicaid billing standards, and others.

Any of a variety of medical and incidental tests and services are within the scope of the disclosure and may make use of the disclosed systems and methods. For example, a person may request dental consultation and services through the platform. A dentist may respond to a request and ship a test kit, a medicine or reagent, or a dental device to the person. Note that the person may be making a request on his or her own behalf, or on behalf of a family member or another person. For example, a person may use a smartphone app to request a medical service on behalf of an elderly or infirm person, and a medical provider may use the backend computer system or order medical transport, or the shipment of a material or device important to the care of the subject. The platform may be used to order, for example, veterinary services. For example, a person could order a test for their dog and have a sample collection kit shipped to the home by use of the smartphone app. Systems and methods of the invention may be particularly beneficial for large animal veterinary services, where it may be difficult to transport, e.g., a horse to the clinic, or where a straightforward prescription refill is requested. In all cases, the user—via the smartphone app (or similar)—may request the medical services from his or her preferred, chosen medical provider or care team, someone with whom they have an existing relationship or are seeking due to reputation or referral.

The system 201 may be used to organize the delivery of wellness products and services that fall outside of the scope of what is licensed by a medical board. For example, the system 201 may be used for the provision of therapeutic services and counseling, e.g., to find and schedule therapy or counseling meetings. The system 201 may be used for the provision of naturopathic medical services, acupuncture, or other non-traditional medical services. The system may be used for engagement with dieticians, optical services, or for ordering of fitting prosthetics. When used for dietary services, the system 201 may be used to request an allergen test. E.g., the person requests a test (e.g., a lateral flow assay or hand-held food testing kit) and his or chosen provider orders the allergen test, which is shipped directly to the person. The system may be used for providing, testing, or fitting ability assistance devices or materials. The system 201 may be used for medical alert services (smartphone app to call provider under duress conditions) or to request transmission of medical information. For example, a person being treated under emergency conditions, or in an unfamiliar location, may use the smartphone app to have his or her regular medical provider send medical history or medication information (e.g., allergies). The system 201 may be used to address industrial or environmental concerns with medical implications, such as tests for asbestos in buildings, or lead in drinking water. As mentioned, the system 201 may be used to engage with veterinary, and in particular, large animal veterinary services, also including home euthanasia and funereal services for animals.

In other embodiments, the system 201 may find particular applicability for people with mobility-related medical needs such as missing or prosthetic limbs, or people that require medical transport services. In fact, the service ordered by a medical provider when the person presses the button on the smartphone app may be medical transport to bring the person to a clinic. The system 201 may find particular applicability in the context of elder care, giving people a very easy mechanism by which to engage their preferred or chosen care providers or care teams. The system 201 may find particular applicability in addressing sexually-transmitted infections or other conditions that involve a social stigma or for which there may be human reluctance to, e.g., publicly visit a pharmacy or purchase a test. In some situations, a person may be reluctant to purchase, in public, a home pregnancy test, and may have his or her preferred, chosen provider order one through use of the smartphone app. The system 201 may find particular applicability in the context of epidemics, and within that, even within the context of institutional requirements. For example, in the case of an epidemic, a large number of tests may need to get shipped rapidly to a large number of people, at a scale that cannot be met by in-person visits. In such cases, the system 201 may be deployed to allow the large number of people to request tests. There may be institutional requires, such as “show a negative Covid test result to come to campus” or similar. The system 201 may be very useful in giving people an easy-to-use tool for complying with such institutional requests.

What is claimed is:

1. A virtual medical service method comprising:
processing, via computer system, a request from a person for a medical service;
sharing the request with a medical provider;
receiving into the computer system an order from the medical provider for at least a portion of the medical service;
informing the person of the order; and
recording a record of the order in the computer system.

2. The method of claim 1, wherein the medical provider is chosen by the person and the person has, with knowledge of the identity of the medical provider, sent the request to the medical provider.

3. The method of claim 1, further comprising recording a plurality of visit records each day for at least several days and periodically transferring the visit records to a billing system computer.

4. The method of claim 1, wherein the request is received when the person uses a smartphone app that transmits the request to the computer system.

5. The method of claim 1, wherein the order from the medial provider includes an order for a test, and the method includes sending instructions to the patient to go to a test site.

6. The method of claim 5, further comprising sending the order to the test site.

7. The method of claim 6, further comprising facilitating the patient and the test site in scheduling the test.

8. The method of claim 5, wherein the patient is instructed to go to the test site and give a sample that includes at least one of blood, breath, and urine.

9. The method of claim 1, wherein the medical provider and the patient conduct a real-time synchronous discussion by text, voice, or video via a digital communications platform and the computer system receives a confirmation of the synchronous discussion and writes the confirmation of the synchronous discussion into the visit record.

10. The method of claim 1, further comprising writing a billing code into the record, optionally wherein the computer system uploads the record to an electronic medical record system.

11. The method of claim 1, further comprising:
authorizing, by the computer system, a laboratory to ship a medical test kit to the person.

12. The method of claim 11, further comprising:
accepting input of a test result from the laboratory; and
providing the person or the medical provider with access to the test result.

13. The method of claim 1, wherein the computer system periodically transmits batches of records to an insurance system.

14. The method of claim 1, wherein the computer system writes the record to an electronic medical records system as a record of a billable visit.

15. The method of claim 1, further comprising initiating shipment of a medical test kit directly to the patient.

16. A system comprising:
at least one processor coupled to a memory subsystem containing instructions executable by the processor to cause the system to obtain a digital patient request for medical service;
share the request with a medical provider;
receive an order from the medical provider for at least a part of the medical service;
inform the patient of the order; and
write a record of the order in the computer system.

17. The system of claim 16, wherein the system writes a billing code into the record.

18. The system of claim 16, wherein the system records a plurality of visit records each day for at least several days and periodically transfers the visit records to a billing system computer.

19. The system of claim 16, wherein the request is received when a patient uses a smartphone app that transmits the request to the computer system.

20. The system of claim 16, wherein the order from the medial provider includes an order for a test, and the system sends instructions to the patient to go to a test site.
